(19) United States
(12) Patent Application Publication
(10) Pub. No.: US 2017/0229225 A1
(43) Pub. Date: Aug. 10, 2017

(54) UNIFORM NANOCOMPOSITIONS, METHODS OF MAKING THE SAME, AND USES OF THE SAME

(71) Applicant: NVIGEN, INC., Sunnyvale, CA (US)

(72) Inventors: Aihua FU, Sunnyvale, CA (US); Chunheng CHENG, Sunnyvale, CA (US)

(21) Appl. No.: 15/500,759

(22) PCT Filed: Aug. 3, 2015
(86) PCT No.: PCT/US2015/043506

§ 371 Date: Jan. 31, 2017

Related U.S. Application Data
(60) Provisional application No. 62/032,567, filed on Aug. 2, 2014.

(51) Int. Cl. H01F 10/12; G01N 33/543; C01G 49/02; A61K 9/14

(52) U.S. Cl. CPC H01F 10/12; A61K 9/143; A61K 47/48861; G01N 33/54346; G01N 33/54326; C01G 49/02; A61K 48/00

(57) ABSTRACT

A uniform cluster of nanocompositions suspended in a liquid media is provided. Methods of making such nanocompositions, and uses of such nanocompositions are also provided. The nanocompositions can be used for nucleic acid extraction and diagnostic assays, for immunoassays, for cell separation, identification and modulation, for controlled functional molecule protection and release, for assays used in the clinic (companion diagnostics) or in the therapeutic development process (drug target validation), and in a system for transcatheter arterial chemoembolization, and demonstrate superior performance due to the uniform property or monodispersity.

### CROSS-REFERENCE TO RELATED APPLICATIONS

The present application claims the benefit of U.S. Provisional Application Ser. No. 62/032,567, filed on Aug. 2, 2014, which is incorporated herein by reference in its entirety.

### FIELD OF THE INVENTION

The present invention generally relates to synthesis of uniform clusters of nano compositions.

### BACKGROUND OF THE INVENTION

Magnetic nanoparticles are of great interest for researchers from a wide range of disciplines, including magnetic fluids, catalysis, biotechnology, biomedicine, magnetic resonance imaging, data storage, and environmental remediation. In particular, superparamagnetic nanopaticles have proved to be very promising for biotechnology/biomedicine applications as they behave as non-magnetic material and remain dispersed when there is no magnetic field while they can show strong magnetic interactions under external magnetic field control. Iron oxide nanoparticles have received the most attention because of their biocompatibility in physiological conditions and low toxicity.

However, it is a technological challenge to control size, shape, stability, and dispersibility of nanoparticles in desired solvents. Several approaches have been developed for synthesizing magnetic iron oxide nanoparticles with controlled size distribution, typically through organometallic processes at elevated temperatures in organic solvents. Additional steps of surface modification are usually performed to transfer the hydrophobic nanoparticles from organic solvent to water for biomedical applications. Furthermore, as these approaches involve reaction mixture of organic solvent at elevated temperature, it is difficult to industrialize and the cost of production is high.

Accordingly, there is a continuing need for magnetic nanoparticles with high uniformity and an efficient and environmental-friendly method for preparing such uniform magnetic nanoparticles.

### BRIEF SUMMARY OF THE INVENTION

The present disclosure provides a uniform cluster of nanocompositions, methods of making such nanocompositions, and uses of such nanocompositions. The nanocompositions can be used in a system for transcatheter arterial chemoembolization.

In one aspect, the present disclosure relates to a composition comprising a uniform cluster of nanocompositions suspended in a liquid media. The nanocompositions as described herein has a mean size that falls into a range between about 1 nm to about 1000 nm (preferably about 1-900 nm, 1-500 nm, 2-400 nm, 5-200 nm, 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 50 nm, 100 nm, 200 nm, 300 nm, 400 nm, 500 nm, 600 nm, 700 nm, 800 nm, 900 nm in size). In certain embodiments, the nanocompositions in the cluster have substantially the same size. In certain embodiments, the size distribution (standard deviation) of the nanocompositions is less than 20%, 15%, 10%, 9%, 8%, 7%, 6%, 5%, 4% or 3% of the mean size of the nanocomposition cluster. In certain embodiments, 70% of the nanocompositions have a size that falls within 20%, 15%, 10%, 9%, 8%, 7%, 6%, 5%, 4% or 3% of the mean size of the nanocomposition cluster. In certain embodiments, the cluster of nanocompositions has a polydispersity index (PDI) less than 0.15 as measured by dynamic light scattering technique. Preferably, the cluster of nanocompositions has a PDI less than 0.1. More preferably, the cluster of nanocompositions has a PDI less than 0.08, 0.07, 0.06, 0.05 or 0.04.

In certain embodiments, the nanocomposition as described in the present disclosure comprises a core nanoparticle and a coating. In certain embodiments, the core nanoparticle is a magnetic nanoparticle or non-magnetic nanoparticle. In certain embodiments, the magnetic nanoparticle is a superparamagnetic iron oxide (SPIO) nanoparticle. In certain embodiments, the SPIO nanoparticle is doped with magnesium, zinc, manganese, cobalt, gold, silver or the combination thereof.

In certain embodiments, the coating is a silanization coating. In certain embodiments, the coating is a surfactant or a polymer. In certain embodiments, the coating contains a functional group. In some preferred embodiments, the functional group is mono-carboxylate acid, di-carboxylate acid, tri-carboxylate acid or tetra-carboxylate acid. In certain embodiments, the functional group is selected from the group consisting of streptavidin, protein A, protein G, antibody, peptide, aptamer, fluorophores, enzymes and drugs. In certain embodiments, the coating is a low density, porous 3-D structure.

In another aspect, the present disclosure provides methods for making uniform cluster of nanocompositions. In an embodiment, the present invention provides a method of making uniform cluster of nanocompositions, comprising (1) mixing a metal salt precursor and a surfactant in an aqueous/alcohol solvent to form a reaction solution; (2) adding a precipitation agent to the reaction solution; (3) obtaining the clusters of nanocompositions; wherein the reaction solution is controlled at a temperature lower than 300 C. Preferably, the reaction solution is controlled at a temperature lower than 200 C. More preferably, the reaction solution is controlled at a temperature lower than 100 C. In another preferred embodiment, the reaction solution does not contain an organic solvent other than alcohol. In another embodiment, the present disclosure provides a composition prepared by a method described herein.

In yet another aspect, the present disclosure provides methods for delivering functional molecules to a tumor tissue by using uniform cluster of nanocompositions. In one embodiment, the delivery is through transcatheter arterial chemoembolization. In another embodiment, the present disclosure provides a system for delivering uniform cluster of nanocompositions through transcatheter arterial chemoembolization.

In anther aspect, the present disclosure relates to a solution for activating nanoparticles used in an application, comprising an acid, a base or a salt. In certain embodiments, the acid is selected from the group consisting of chloric acid, sulfuric acid, sulfurous acid, phosphonic acid, phosphorous acid, carboxylic acid, and amino acid, and combinations thereof. In certain embodiments, the base is selected form the group consisting of sodium hydroxide, ammonium hydroxide, potassium hydroxide, tetramethylammonium hydroxide, and combinations thereof. In certain embodiments, the salt is selected from the group consisting of Tris chloride, sodium carboxylate, ammonium carboxylate, sodium sulfate, sodium alkyl sulfate and combinations thereof. In certain embodiments, the solution further comprising polyethylene glycol, tween, chaps, propylene glycol, butylene glycol, salt, glycerol, sucrose, deoxyribonucleotide, small peptides, or proteins.

In another aspect, the present disclosure relates to a method of using nanocompositions in an application, comprising providing a cluster of nanocompositions suspended in a liquid media; and adding to the cluster a solution to activate the nanocompositions for the application.

### BRIEF DESCRIPTION OF THE FIGURES

FIG. 1. Nanocompositions prepared by the procedure described in Example 1.

FIG. 2. Metal doped magnetic nanocompositions prepared using the method disclosed in the present disclosure.

FIG. 3. Monodispersed nanocompositions are water-soluble, so they are completely dispersed in the water phase. No nanocompositions are observed in the top phase consisting of Hexane.

FIG. 4. Transmission electron microscopy image for nanocomposition clusters prepared by the method disclosed in the present disclosure. They demonstrate monodispersity as measured by dynamic light scattering measurement.

FIG. 5. Uniform magnetic nanocompositions can be used for DNA size fragment selection with cleaner cut off.

FIG. 6. Uniform magnetic nanocompositions can associate with antibody and applied for antibody purification and immunoassays. The monodispersity of the nanocompositions result in more consistent assay data.

FIG. 7. Illustration of the apparatus for utilizing nanocompositions to deliver chemotherapy and collect excess chemodrugs. The apparatus comprises two catheters. One catheter is inserted in the artery supplying the tumor in the organ, for example, through a hepatic artery branch. Nanocompositions are injected from the catheter or a container associated with the catheter, and directed to the tumor. The other catheter is inserted in the hepatic vein, with a magnetic structure that can be extended outside the catheter opening after introduction. The magnetic structure can collect excess nanocompositions with drugs through magnetic attraction. The magnetic structure can also be magnetic structures deposited onto a filtration material, to improve the collection of excess nanocompositions with chemodrugs using filtration material alone.

FIG. 8A. Emulsion solution containing perfluorocarbon and uniform magnetic nanocompositions observed under microscope

FIG. 8B. Nanobubble emulsion solution observed under microscope. Various size of bubbles were created due to physical shaking of the emulsion.

### DETAILED DESCRIPTION OF THE INVENTION

Before the present disclosure is described in greater detail, it is to be understood that this disclosure is not limited to particular embodiments described, and as such may, of course, vary. It is also to be understood that the terminology used herein is for the purposes of describing particular embodiments only, and is not intended to be limiting, since the scope of the present disclosure will be limited only by the appended claims. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, solid state chemistry, inorganic chemistry, organic chemistry, physical chemistry, analytical chemistry, materials chemistry, biochemistry, biology, molecular biology, recombinant DNA techniques, pharmacology, imaging, and the like, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” series (Academic Press, Inc., 1955-2014); “Current Protocols in Molecular Biology” (F. M. Ausubel et al., eds., 1987, and periodic updates); “PCR: The Polymerase Chain Reaction”, (Mullis et al., eds., 1994). Primers, polynucleotides and polypeptides employed in the present disclosure can be generated using standard techniques known in the art.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

The following embodiments are put forth so as to provide those of ordinary skill in the art with a complete disclosure and description of how to perform the methods and use the nanostructure disclosed and claimed herein. Efforts have been made to ensure accuracy with respect to numbers (e.g., amounts, temperature, etc.), but some errors and deviations should be accounted for.

It must be noted that, as used in the specification and the appended claims, the singular forms “a,” “an,” and “the” include plural forms of the same unless the context clearly dictates otherwise. Thus, for example, reference to “a compound” includes a plurality of compounds. In this specification and in the claims that follow, reference will be made to a number of terms that shall be defined to have the following meanings unless a contrary intention is apparent.

The present disclosure provides a uniform cluster of nanocompositions, methods of making such nanocompositions, and uses of such nanocompositions.

Uniform Cluster of Nanocompositions

In one aspect, the present disclosure relates to a composition comprising a uniform cluster of nanocompositions suspended in a liquid media.

The term “uniform nanocompositions” or “uniform cluster of nanocompositions” as used herein refers to a plurality of nanocompositions that have substantially the same size, shape or mass.

In certain embodiments, the cluster of nanocompositions as described herein has a mean size or diameter that falls with a range between about 1 nm to about 1000 nm (preferably about 1-900 nm, 1-500nm, 2-400 nm, 5-200 nm, 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 50nm, 75nm, 100nm, 125 nm, 150 nm, 175 nm, 200 nm, 300 nm, 400 nm, 500 nm, 600 nm, 700 nm, 800 nm, 900 nm in size). Methods of synthesizing uniform cluster of nanocompositions with controlled size are disclosed in the present application.

In certain embodiments, a cluster of nanocompositions is uniform when any two nanocompositions in the cluster have substantially the same size. In certain embodiments, the cluster of the nanocompositions is uniform when the nanocompositions have a size distribution (i.e., standard deviation of the sizes of the nanocompositions) less than 20%, preferably 15%, 10%, more preferably 9%, 8%, 7%, 6%, 5%, 4% or 3% of the mean size of the cluster. In certain embodiments, the cluster of the nanocompositions is uniform when 70% of the nanocompositions have a size that falls within 20%, preferably 15%, 10%, preferably 9%, 8%, 7%, 6%, 5%, 4% or 3% of the mean size of the nanocomposition cluster. In certain embodiments, the cluster of nanocompositions has a polydispersity index (PDI) less than 0.15 as measured by dynamic light scattering technique. Preferably, the cluster of nanocompositions has a PDI less than 0.1. More preferably, the cluster of nanocompositions has a PDI less than 0.08, 0.07, 0.06, 0.05 or 0.04.

As used herein, polydispersity index (PDI) is a measure of the distribution of sizes of nanocompositions in a mixture. A collection of nanocompositions is uniform if the nanocompositions have substantially the same size, shape or mass. One conventional method of measuring nanoparticle size and size distribution is using dynamic light scattering (DLS) technology, which is a technique to determine the size distribution of small particles in suspension. Detailed mechanism and application of dynamic light scattering can be found at Berne, B. J. and Pecora, R., Dynamic Light Scattering. Courier Dover Publications (2000), which in incorporated herein by reference. In this patent application, the related DLS measurements are performed on a Brookhaven Nanosizer.

In certain embodiments, the nanocomposition as described herein comprises a core nanoparticle and a coating.

The core nanoparticles as described in the present disclosure can be a magnetic nanoparticle or a non-magnetic nanoparticles. In certain preferred embodiments, the magnetic nanoparticle is a superparamagnetic iron oxide (SPIO) nanoparticle. In certain embodiments, the SPIO nanoparticle is doped with magnesium, zinc, manganese, nickle, cobalt, cadmium, gold, silver or the combination thereof.

The SPIO nanoparticle is an iron oxide nanoparticle, either maghemite (γ-Fe₂O₃) or magnetite (Fe₃O₄), or nanoparticles composed of both phases. Nanoparticles are said to be in the superparamagnetic state in that their magnetization appears to be in average zero in the absence of an external magnetic field, while the nanoparticles can be magnetized by an external magnetic field. Methods to synthesize a uniform cluster of SPIO nanoparticles are disclosed in the present application.

The non-SPIO nanoparticles include, for example, metallic nanoparticles (e.g., gold or silver nanoparticles (see, e.g., Hiroki Hiramatsu,F. E. O., Chemistry of Materials 16, 2509-11 (2004)), semiconductor nanopaticles (e.g., quantum dots with individual or multiple components such as CdSe/ZnS (see, e.g., M. Bruchez, et al., Science 281, 2013-16 (1998))), doped heavy metal free quantum dots (see, e.g., Narayan Pradhan et al., J. Am, Chem. Soc 129, 3339-47 (2007)) or other semiconductor quantum dots); polymeric nanoparticles (e.g., particles made of one or a combination of PLGA (poly(lactic-co-glycolic acid) (see, e.g., Minsoung Rhee et al., Adv. Mater. 23, H79-83 (2011)), PCL (polyacprolactone) (see., e.g., Marianne Labet et al., Chem. Soc. Rev. 38, 3484-3504 (2009)), PEG (polyethylene glycol) or other polymers); siliceous nanoparticles, and non-SPIO magnetic nanoparticles (e.g., MnFe204 (see, e.g., Jae-Hyun Lee et al., Nature Medicine 13, 95-99 (2006)), synthetic antiferromagnetic nanoparticles (SAF) (see, e.g., A. Fu et al., Angew. Chem. Int. Ed. 48, 1620-24 (2009)), and other types of magnetic nanoparticles). The size of the core nanoparticle ranges from about 1 nm to about 900 nm (preferably 1-500 nm, 2-400 nm, 5-200 nm, 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 50 nm, 75 nm, 100 nm, 125 nm, 150 nm, 175 nm, 200 nm).

In certain embodiments, the core nanoparticle has a shape of sphere, rod, tetrapod, pyramidal, multi-armed, nanotube, nanowire, nanofiber, or nanoplate.

Methods of synthesizing uniform clusters of nanocompositions with non-SPIO core nanoparticles are disclosed in the present application.

As used herein, the term “coating” refers any substance in which at least one core nanoparticle can be embedded. Any suitable coatings known in the art can be used, for example, a surfactant, a polymer coating and a non-polymer coating. The coating interacts with the core nanoparticles through 1) intra-molecular interaction such as covalent bonds (e.g., sigma bond, pi bond, delta bond, double bond, triple bond, quadruple bond, quintuple bond, sextuple bond, 3c-2e bond, 3c-4e bond, 4c-2e bond, agostic bond, bent bond, dipolar bond, pi backbond, conjugation, hyperconjugation, aromaticity, hapticity, and antibonding), metallic bonds (e.g., chelating interactions with the metal atom in the core nanoparticle), or ionic bonding (cation πr-bond and salt bond), and 2) inter-molecular interaction such as hydrogen bond (e.g., dihydrogen bond, dihydrogen complex, low-barrier hydrogen bond, symmetric hydrogen bond) and non covalent bonds (e.g., hydrophobic, hydrophilic, charge-charge, or π-stacking interactions, van der Waals force, London dispersion force, mechanical bond, halogen bond, aurophilicity, intercalation, stacking, entropic force, and chemical polarity).

In certain embodiments, the coating is a silanization coating. In an embodiment, the silanization coating is a coating including silane and/or silane-like molecules (or the reaction products of those molecules with the surface) onto the surface of the SPIO nanoparticles.

The coating can be amorphous. The thickness of the coating can be controlled so that coated nanoparticles can be created for particular applications. In an embodiment, the silanization coating is made by cross-linking of trimethoxyl silanes with appropriate functional groups, such as a mercapto group, an amino group, a mercapto/amino group, a carboxyl group, a phosphonate group, an alkyl group, a polyethylene oxide group (PEG), and combinations thereof.

In certain embodiments, the coating is a surfactant. In certain embodiments, the surfactant is a compound containing carboxylate, sulfonate, sulfate, phosphate, hydrogen, amine, ammonium, betaine and sulfobetaine groups.

In certain embodiments, the coating is a compound containing carboxylate, sulfonate, sulfate, phosphate, hydrogen, amine, ammonium, betaine and sulfobetaine groups.

In certain embodiments, the coating is a polymer. Examples of polymer include, but not limited to a polypeptide that may be optionally functionalized with various side groups. The polymer coating can be chosen from the group consisting of chitosan, polystyrene, polyethyleneglycol, polypropylene glycol, polymethacrylate, polyacrylate, polyacrylamide, polyaldehyde, dextran, sucrose, polysaccharide, agarose.

In certain embodiments, the coating contains one or more functional groups. Examples of the functional group include, but are not limited to amino, mercapto, mono-carboxylate acid, di-carboxylate acid, tri-carboxylate acid or tetra-carboxylate acid, streptavidin, avidin, protein A, protein G, antibody, peptide, aptamer, fluorophores, enzymes and drugs.

The functional groups may be introduced during the formation of the coating, for example, by adding silicon-containing compounds containing such functional groups during a cross linking process. The functional groups may also be introduced after the formation of the coating, for example, by introducing functional groups to the surface of the coating by chemical modification. In certain embodiments, the functional groups are inherent in the coating.

In certain embodiments, the coating is a low density, porous 3-D structure, as disclosed in WO2013112643, which is incorporated herein in its entirety. The low density, porous 3-D structure refers to a structure with density at least 10 times lower than existing mesoporous materials (e.g., mesoporouos materials having a pore size ranging from 2 to 50 nm). In certain embodiments, the low density, porous 3-D structure has a density of <1.0 g/cc (e.g., 0.01 mg/cc to 1000 mg/cc).

The cluster of nanocomposition as described herein keeps uniformity and stability when it is suspended in a liquid media. In certain embodiments, the nanocomposition is soluble in the liquid media, i.e., the nanocomposition is stable and dispensable in the liquid media. The nanocomposition suspended in the liguid media does not aggregate or precipitate. The liquid media used to suspend nanocompositions include, but not limited to water, a biological buffer (e.g., PBS, TBS), alcohol, and a combination thereof.

Methods of Preparation

In another aspect, the present disclosure provides methods for making a uniform cluster of nanocompositions. It has been a technological challenge to control size, shape, stability, and dispersibility of nanocompositions in desired solvents. Several approaches have been developed for synthesizing magnetic iron oxide nanoparticles with controlled size distribution, typically through organometallic processes at elevated temperatures in organic solvents (see, e.g., US20080032132). Additional steps of surface modification are usually performed to transfer the hydrophobic nanoparticles from organic solvent to water for biomedical applications. However, as these approaches involve reaction mixture of organic solvent at elevated temperature, it is difficult to industrialize and the cost of production is high. One of the surprising discoveries of the instant invention is a method for preparing uniform cluster of nanocompositions that are dispensable or water-soluble under mild preparation conditions (aqueous/alcohol solvents and relatively low temperature).

In an embodiment, the method of making the uniform cluster of nanocompositions comprises (1) mixing a metal salt precursor and a surfactant in an aqueous/alcohol solvent to form a reaction solution; (2) adding a precipitation agent to the reaction solution; (3) obtaining the cluster of nanocompositions; wherein the reaction solution is controlled at a temperature lower than 300° C. Preferably, the reaction solution is controlled at a temperature lower than 200° C. More preferably, the reaction solution is controlled at a temperature lower than 100° C.

The metal salt precursors include, but are not limited to, iron salt, magnesium salt, zinc salt, cadmium salt, manganese salt, nickel salt, cobalt salt, gold salt, silver salt in the form of chloride, sulfate, nitrate, fluoride, bromide, iodide, sulfide, selenide, telluride, acetate, oxalate, citrate or phosphate.

In certain embodiments, the metal salt precursor is a mixture of iron (II) salt and iron (III) salt. The iron (II) salt include iron (II) chloride, iron (II) sulfate, iron (II) nitrate, iron (II) fluoride, iron (II) bromide, iron (II) iodide, iron (II) sulfide, iron (II) selenide, iron (II) telluride, iron (II) acetate, iron (II) oxalate, iron (II) citrate and iron (II) phosphate. The iron (III) salt include of iron (III) chloride, iron (III) sulfate, iron (III) nitrate, iron (III) fluoride, iron (III) bromide, iron (III) iodide, iron (III) sulfide, iron (III) selenide, iron (III) telluride, iron (III) acetate, iron (III) oxalate, iron (III) citrate and iron (III) phosphate.

In certain embodiments, the mixture of metal salt precursors also includes non-iron metals such as cobalt, nickel, magnesium, manganese, zinc, gold and silver in corresponding salt forms. In such case, these non-iron metals can be incorporated into the synthesis so that the final products are iron based complex oxides.

Suitable surfactants for use in the method of the present disclosure can be chosen from a wide range of polyelectrolytes such as, but not limited to those containing carboxylate groups including polyacrylic acid and polymethacrylic acid, citric acid, tartaric acid, lactic acid, acetic acid, oxalic acid, propionic acid, butyric acid, oleic acid, valeric acid, caproic acid, enanthic acid, tannic acid, capryllic acid, pelargohic acid, pelargohic acid, capric acid, undecyllic acid, laruric acid, tridecylic acid, myristic acid, palmitic acid, margaric acid, stearic acid, arachidic acid, and those containing sulfonate, sulfate, phosphate, amine, ammonium, betaine, or sulfobetaine groups.

Suitable alcohol for use in the method of the present disclosure can be chosen from alcohol that has 1, 2, 3, 4, 5, 6, 7, 8, 9, 10, 11, 12 or more carbon atoms. In certain embodiements, the alcohol can be monohydric alcohol, or polyhydric alcohol. Illustrative examples of monohydric alcohols include methanol, ethanol, propanol, butanol, pentanol, hexyl alcohol, etc. Illustrative examples of polyhydric alcohols include propylene glycol, glycerol, threitol, xylitol, etc.

In certain embodiments, the alcohol can have a saturated carbon chain or an unsaturated carbon chain. An alcohol having a saturated carbon chain can be represented as CₙH₍₂ₙ₊₂₎O in chemical formula. In certain embodiments, n is no less than 3, or no less than 4, or no less than 5 (e.g., n=3, 4, 5, 6, 7, 8, 9, 10, 11, 12 or more). Alcohol with an unsaturated carbon chain has a double or a triple bond between two carbon atoms. In certain embodiments, the alcohol can be a cyclic alcohol, for example, cyclohexanol, inositol, or menthol.

In certain embodiments, the alcohol can have a straight carbon chain (e.g., n-propyl alcohol, n-butyl alcohol, n-pentyl alcohol, n-hexyl alcohol, etc) or a branched carbon chain (e.g., isopropyl alcohol, isobutyl alcohol, tert-butyl alcohol, etc). In certain embodiments, the alcohol is present in a volume fraction of about 30% to about 70% (e.g., about 30% to about 70%, about 30% to about 60%, about 30% to about 55%, about 40% to about 70%, about 45% to about 70%, about 40% to about 60%). In certain embodiments, the alcohol is present in volume fraction of around 50% (e.g., around 45%, around 46%, around 47%, around 48%, around 49%, around 50%, around 51%, around 52%, around 53%, around 54%, around 55%, around 56%, around 57%, around 58%, around 59%, or around 60%,).

In another preferred embodiment, the reaction solution does not contain an organic solvent other than alcohol. The organic solvents other than alcohol include, but not limited to toluene, chloroform, hexane.

In the method of the present disclosure, the precipitation of the cluster of nanocompositions can be initiated by adding a precipitation agent. The precipitation agent include, but not limited to bases such as metal hydroxides, carbonates, bicarbonates, phosphates, hydrogen phosphate, dihydrogen phosphates of group 1 and 2, ammonium (for example, NaOH, KOH, NH4OH, Na₂CO₃, K₂CO₃), tetramethyl ammonia hydroxide, ammonia, as well as group 1 salts of carbanions, amides and hydrides.

Products by Process

Another aspect of the present disclosure relate to a cluster of nanocompositions prepared by any of the methods provided herein. The nanocompositions prepared herein may be optionally isolated, purified or dried using methods described herein and/or conventional methods known in the art.

Methods of Use

In yet another aspect, the present disclosure provides the use of the uniform cluster of nanocompositions described herein. The use of the uniform cluster of nanocompositions include, but not limited to manufacture of therapeutic or diagnostic composition, manufacture of reagents useful in a qualitative or quantitative tests, manufacture of a reagent useful in molecular imaging, and manufacture of a reagent useful in separation, purification or enrichment. The uniform cluster of nanocompositions of the present disclosure can also be used for encapsulating or protecting functional molecules, such as drugs, or be used as a carrier for functional molecules. The uniform cluster of nanocompositions of the present disclosure can also be applied to targeted delivery or controlled release of functional molecules.

In certain embodiments, the uniform cluster of nanocompositions are used for interacting with nucleic acid for extraction, size selection, diagnostic assays, and obtained better results because of the monodispersity of the nanocompositions.

In certain embodiments, the uniform cluster of nanocompositions are used for immunoassay, and obtained better results such as consistency and better quantification because of the monodispersity of the nanocompositions.

In certain embodiments, the uniform cluster of nanocompositions are used for cell separation, identification and modulation experiment, and obtained better results because of the monodispersity of the nanocompositions, such as quantitative identification of different cell types based on cell surface marker interaction with the uniform nanocompositions, better cell sorting and differentiation either through fluorescent signal or magnetic property of tagged uniform particles on cell surface, or more consistent stimulation of cell behavior from the uniform nanocompositions.

In certain embodiments, the uniform cluster of nanocompositions are used for better diagnostic assays or processing clinical samples because of the uniformity of the nanocompositions. The uniform cluster of nanocompositions are able to provide more consistent and reliable data, for example, in target validation for therapeutic development, or for companion diagnostics to detect cancer at the earliest stage or for prognosis evaluation after treatment.

In certain embodiments, the uniform cluster of nanocompositions of the present disclosure can be applied to systematic or focused delivery of functional molecules, such as chemotherapy.

In one embodiment, the focused delivery of functional molecules is through transcatheter arterial chemoembolization (TACE). In one example, uniform cluster of magnetic nanocompositions that carry chemotherapeutic agents are administrated to the tumor tissue through TACE. The nanocompositions prevent the chemotherapeutic agents from being washed away from the tumor tissue after embolization. In a preferred embodiment, excess nanocompositions with chemotherapeutic agents are collected with a magnetic stand and removed out of the body to reduce toxic side effects.

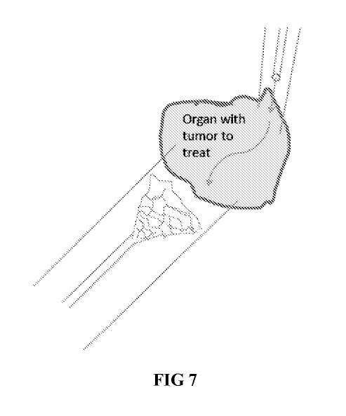

In another aspect, the present disclosure provides an apparatus for delivering uniform cluster of nanocompositions through TACE. In certain embodiments, the apparatus comprises two catheters: one catheter comprises an injectable container that holds the solution of nanocomposition-chemodrugs inside the catheter, for injecting nanocomposition-chemodrug embolization into the targeted tissue or organ site; the other catheter holds a magnetic structure, which can extrude outside the catheter once in position to collect the excess nanocomposition-chemodrug embolization. The magnetic structure can be a permanent magnetic stand, a magnetizable magnetic mesh structure, or an electromagnet that can be switched on and off to generate needed magnetic forces to attract the excess nanocomposition-chemodrug embolization from a location outside the body (see FIG. 7 for an illustration of the set up).

The following examples are presented to illustrate the present invention. They are not intended to limiting in any manner.

### EXAMPLE 1

The following is an example of preparation and characterization of a uniform cluster of SPIO nanocompositions.

0.5 g of FeCl₂ in 5 ml diH₂O and 1.0 g of FeCl₃ in 5 ml H₂O were mixed in a 250 ml reaction flask with 3 inlets. The flask was sonicated in a sonicator filled with water between 65 and 70 degree ° C. and purged with N₂ for about 10 minute. A base-mix was prepared by dissolving 80 mg of lauryl acid in isopropyl alcohol, followed by adding 80 mg of oleic acid. Just before adding the base-mix into the flask, 15 ml of 30% NH₄OH was added to the acid and oleic acid mixture. After adding the base-mix, the flask was sonicated with heating and N₂ purging for another 10 minutes, before stopping the N₂ purging. Then the flask sonicated without N₂, for 20 minutes. The flask was removed from the sonicator, and cooled down for 5 to 15 minutes.

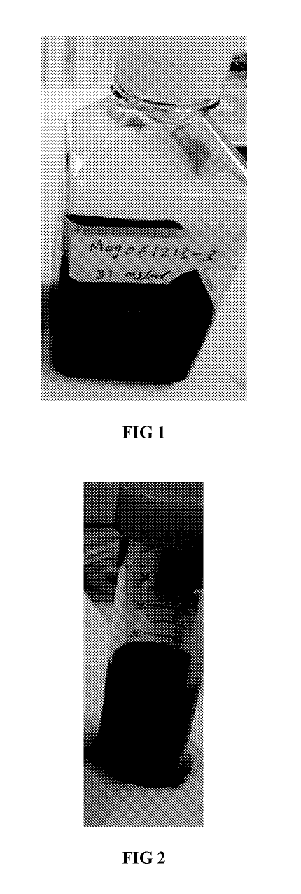

The crude in the flask was transferred and rotated on a rotarack for at least 2 hours. The crude was washed for 5 times, with first three times about 30 ml isopropyl alcohol, two times diH₂O. The washed beads were checked under microscope before transferred into a clean container (see FIG. 1). Size selection was performed when necessary.

The size of the nanocompositions were controlled by controlling the quantities of different ingredients in the nanocomposition reaction, or the coating thickness that can be tuned by controlling the coating material quantity.

As shown in FIG. 2, nanocompositions doped with other metal elements could be prepared with similar methods.

Nanocompositions with different sizes from 100 nanometer to 1 um, and with different surface coatings/molecules/functions were prepared.

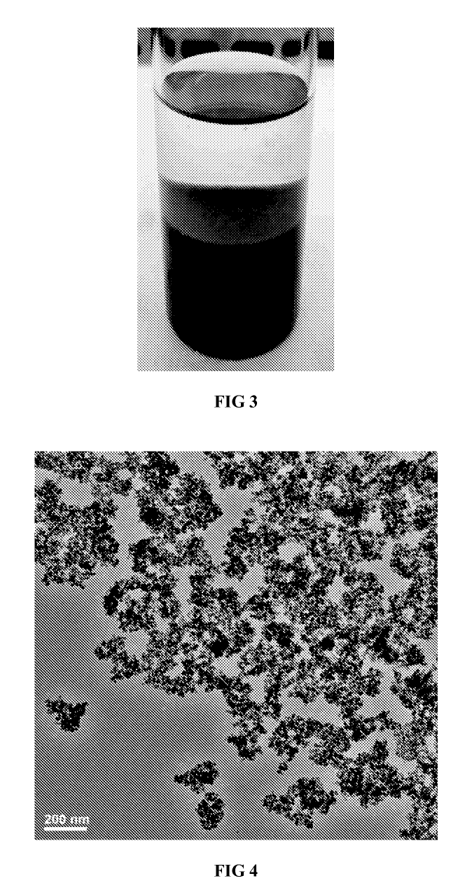

As illustrated in FIG. 3, the prepared nanocompositions could be dispersed in water solutions.

The size distribution of the nanocompositions was measured using a BrookHaven NanoSizer. As illustrated in Table 1, polydispersity index of the nanocompositions was as small as <0.05, which is around the limit of the dynamic scattering instrument.

TABLE 1
Mean size and size distribution of nanocomposition clusters

| Batch | Mean Size (nm) | PDI |
|---|---|---|
| 1 | 230.1 | 0.143 |
| 2 | 346.2 | 0.032 |
| 3 | 1135.9 | 0.097 |
| 4 | 251.1 | 0.064 |

FIG. 5 showed the nanocomposition clusters using transmission electron microscopy. These nanocomposition cluster formed could go through the silanization coating, and demonstrate monodispersity as shown in Table 1 using dynamic light scattering experiment.

### EXAMPLE 2

The following is an example of using the uniform cluster of nanocompositions in isolating DNA.

1,000ng DNA Ladder of 100-1,000 bp (Fisher Scientific) were mixed with 20 ul nanocompositions at room temperature for 30 minutes. The nanocompositions were pelleted on magnet stand and washed with 100 ul fresh 70% ethanol twice. The captured ladder were eluted and analyzed on 3% agrose gel. As illustrated in FIG. 4, the magnetic nanocomposition showed clean cut off in DNA size fragment selection. Comparison with other products on market, using the uniform magnetic nanocomposition have consistently generated DNA libraries with better clean up results.

### EXAMPLE 3

The following is an example of using the uniform cluster of nanocompositions in binding antibodies.

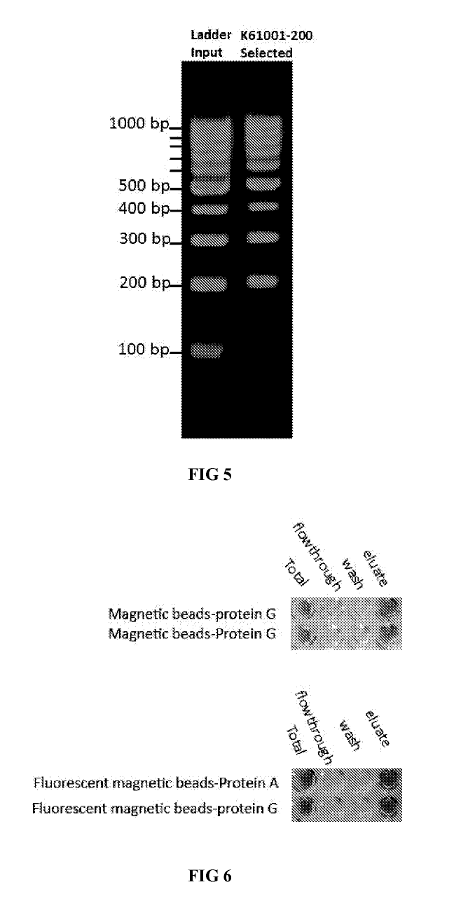

As shown in FIG. 6, uniform nanocompositions composed of either only magnetic nanoparticles or with both magnetic and fluorescent properties were applied for protein capturing assays. The nanocomposites were conjugated with protein A or protein. The conjugated nanocomposites were applied to capture antibodies from a solution. As shown in example 6, duplicate experiments were performed using 10 ug of protein A conjugated nanocomposites to capture 1 ug of antibody in solution. After magnetic separation, the uniform nanocomposition demonstrated more consistent and reproducible results. This feature is very important for clinical immunodiagnostic assays.

### EXAMPLE 4

The uniform cluster of nanocompositions can be used for control and release of functional molecules, such as proteins, nucleic acids, signal generating molecules, drugs. The following example used uniform cluster of nanocompositions for control and release of DNA.

Two magnetic nanocomposition samples (Sample 1 and Sample 2), measured with 80 ng of beads solution, were mixed with DNA of 10 ul at a concentration of 50 μg/ml for 30 minutes in pH 8 buffer (tris, PEG 8000, NaCl). The resulting materials were washed 2 times with 100 ul of 70% ethanol, and then air-dried for 5 minutes. To the dry material in the tube was added 20 ul elution solution: Tris buffer containing 10 mM NaCl. The original supernatant of the solution after magnetic beads absorption, reflecting non-absorbed DNA quantity onto magnetic nanocompositions, the resulting eluting DNA after 5 min and 10 days and standard DNA was measured by a fluorescence plate reader using a Pico green dye. The percentage release was calculated using a linear fitting curve for the DNA control samples. As shown in Table 2, the uniform nanocompositions absorbed over 90% of the DNA after 30 min absorption.

Fluorescence reading of standard DNA control samples at 20%, 60% and 100% quantity in 20 μl solution are: 569, 1488 and 1606.

TABLE 2
The non-absorbed DNA quantity and releasing
results at different time points

| | Percentage of DNA (Fluorescence reading) | | |
|---|---|---|---|
| | | Sup reading after | Sup reading after |
| | Sup reading | adding 20 μl of | adding 29 μl of |
| | after 30 min | elution buffer | elution buffer |
| Nanocomposition | Absorption | for DNA release | for DNA release |
| sample | of DNA | in 5 mins | after 10 days |
| Sample 1 | <10% (173) | 20% (639) | 80% (1458) |
| Sample 2 | <10% (149) | 30% (863) | 70% (1430) |

### EXAMPLE 5

The following is an example of using uniform nanocomposition described herein to prepare perfluorocarbon emulsion.

An stable emulsion were prepare by combining the following ingredients:

(A) perfluorocarbon liquid, which has high solubility of oxygen and can be used to carry oxygen in the body;

(B) aqueous solution including the combination of 6 ingredients in PH8;

(C) vegetable oils such as sunflower oil, olive oil, avocado oil, and canola oil;

(D) uniform nanocompositions.

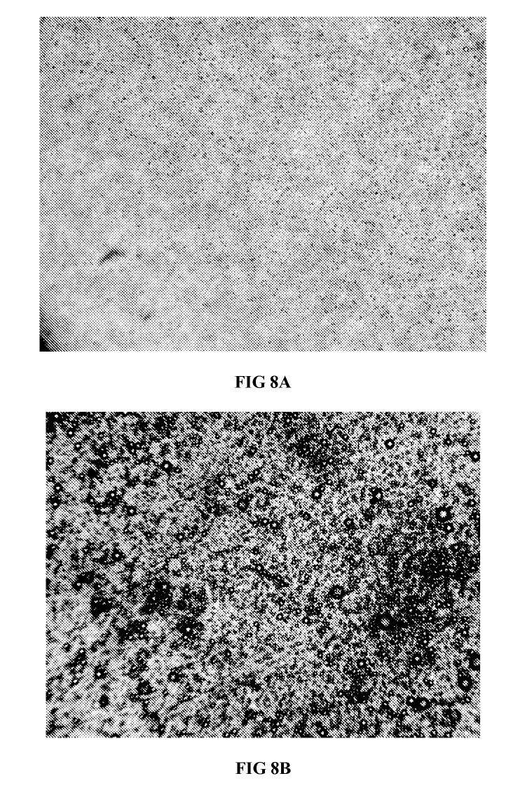

The emulsion formed as a light brown homogeneous slight viscous liquid, which was stable at room temperature and 4 degree. As shown in FIG. 8A and FIG. 8B, the magnetic particles were well dispersed in the emulsion solution, and some bubbles were observed after shaking the emulsion.

Two formulations of nanobubbles were diluted for 6 times by water or 20 times by 0.02% SDS solution, respectively. Sizes and distribution of these two diluted formulations of nanobubbles with PFC and magnetic nanocompositions were measured with dynamic scattering (DLS) technology. As shown in Table 3, the emulsion had narrow particle size and particle size distribution.

The emulsion stabilized perfluorocarbon from 25% up to 40% and it was stable as homogenous solution containing up to 15 mg/ml of nanocompositions.

TABLE 3
Size and distribution of nanobubble emulsion.

| | Combined nanobubble | # number based | |
|---|---|---|---|
| | size (nm) | nanobubble size (nm) | PDI |
| Sample 1 in water | 409.4 | 157.4 | 0.231 |
| Sample 1 in SDS | 408.4 | 247.2 | 0.162 |
| Sample 1 in water | 377.9 | 133.8 | 0.251 |
| Sample 2 in SDS | 469.3 | 173.3 | 0.240 |

While the invention has been particularly shown and described with reference to specific embodiments (some of which are preferred embodiments), it should be understood by those having skill in the art that various changes in form and detail may be made therein without departing from the spirit and scope of the present invention as disclosed herein.

1. A composition comprising a cluster of nanocompositions suspended in a liquid media, said cluster of nanocompositions having a mean size and a size distribution, wherein the mean size falls into a range between about 1 nm to about 1000 nm and the size distribution is within about 20% of the mean size, wherein each of the nanocompositions comprises a core nanoparticle and a coating.

2. The composition of claim 1, wherein the cluster of nanocompositions has a polydispersity index (PDI) less than 0.15 as measured by dynamic light scattering technique.

3. The composition of claim 1, wherein the core nanoparticle comprises a magnetic nanoparticle, a non-magnetic nanoparticle or a combination thereof.

4. The composition of claim 1, wherein the core nanoparticle is a superparamagnetic iron oxide (SPIO) nanoparticle.

5. The composition of claim 4, wherein the SPIO nanoparticle is doped with magnesium, zinc, manganese, cobalt, gold, silver or the combination thereof.

6. The composition of claim 3, wherein the non-magnetic nanoparticle comprises a gold, silver, graphene, polystyrene, semiconductor nanoparticle or a combination thereof.

7. The composition of claim 1, wherein the coating is a silanization coating, a surfactant or a polymer coating.

8. (canceled)

9. The composition of claim 1, wherein the coating comprises a ligand selected from the group consisting of mono-, di-, tri-, or tetra-sulfate, sulfonate, sulfite, phosphonate, carboxylate, amino acid, or a combination thereof.

10. (canceled)

11. The composition of claim 1, wherein the coating is a low density, porous 3-D structure.

12. The composition of claim 1, wherein the coating comprises a functional molecule selected from a group consisting of chromogenic substrate, streptavidin, protein A, protein G, antibody, peptide, aptamer, fluorophores, enzymes and drugs.

13. (canceled)

14. The composition of claim 1, wherein the liquid media is water, PBS, TRIS buffer, alcohol, or a mixture of water and alcohol.

15. The composition of claim 1, further comprising a perfluorcarbon liquid.

16. A method of producing a uniform cluster of nanocompositions, comprising:
mixing a metal salt precursor and a surfactant in an aqueous/alcohol solvent to form a reaction solution;
adding a precipitation agent and a surfactant to the reaction solution;
obtaining the cluster of nanocompositions;
wherein the reaction solution is controlled at a temperature lower than 300 degree C.

17. The method of claim 16, wherein the metal salt precursor comprises an iron (II) salt precursor and an iron (III) salt precursor.

18. The method of claim 17, wherein the iron (II) salt precursor is selected from the group consisting of iron (II) chloride, iron (II) sulfate, iron (II) nitrate, iron (II) fluoride, iron (II) bromide, iron (II) iodide, iron (II) sulfide, iron (II) selenide, iron (II) telluride, iron (II) acetate, iron (II) oxalate, iron (II) citrate and iron (II) phosphate, and the iron (III) salt precursor is selected from the group consisting of iron (III) chloride, iron (III) sulfate, iron (III) nitrate, iron (III) fluoride, iron (III) bromide, iron (III) iodide, iron (III) sulfide, iron (III) selenide, iron (III) telluride, iron (III) acetate, iron (III) oxalate, iron (III) citrate and iron (III) phosphate.

19. The method of claim 17, wherein the metal salt precursor further comprises a non-iron metal salt precursor.

20. The method of claim 19, wherein the non-iron metal salt precursor is selected from the group consisting of magnesium, zinc, manganese, cadmium, cobalt, gold, and silver in the form of chloride, sulfate, nitrate, fluoride, bromide, iodide, sulfide, selenide, telluride, acetate, oxalate, citrate, phosphate, or chloroauric acid.

21. The method of claim 16, wherein the surfactant is a compound containing carboxylate, sulfonate, sulfate, phosphate, hydrogen, amine, ammonium, betaine and sulfobetaine groups.

22. The method of claim 16, wherein the reaction solution does not contain an organic solvent other than alcohol.

23. (canceled)

24. (canceled)

25. (canceled)

26. A composition prepared by the method of claim 16.

27. (canceled)

28. (canceled)
